(19) United States
(12) Patent Application Publication
(10) Pub. No.: US 2023/0233345 A1
(43) Pub. Date: Jul. 27, 2023

(54) STENT ANTENNA AND MEDICAL DATA COMMUNICATION APPARATUS

(71) Applicant: IUCF-HYU (INDUSTRY-UNIVERSITY COOPERATION FOUNDATION HANYANG UNIVERSITY), Seoul (KR)

(72) Inventor: Hyoungsuk YOO, Suwon-si (KR)

(73) Assignee: IUCF-HYU (INDUSTRY-UNIVERSITY COOPERATION FOUNDATION HANYANG UNIVERSITY), Seoul (KR)

(21) Appl. No.: 18/153,699

(22) Filed: Jan. 12, 2023

(30) Foreign Application Priority Data
Jan. 25, 2022 (KR) 10-2022-0010514

(51) Int. Cl. A61F 2/82; A61B 5/00

(52) U.S. Cl. CPC A61F 2/82; A61B 5/6862; A61B 5/02158

(57) ABSTRACT

Disclosed herein is a stent antenna that is inserted into a body and used, including a main branch having a mesh shape, a plurality of branched branches which are branched off from the main branch and each have a mesh shape, and a feed line connected to the main branch to supply power to the stent antenna.

### CROSS-REFERENCE TO RELATED APPLICATION

This application claims priority to and the benefit of Korean Patent Application No. 10-2022-0010514, filed on Jan. 25, 2022, the disclosure of which is incorporated herein by reference in its entirety.

### BACKGROUND

### 1. Field of the Invention

The present disclosure generally relates to a stent antenna and a medical data communication apparatus.

### 2. Discussion of Related Art

A stent refers to a cylindrical medical material used to normalize the flow of blood or body fluids in blood vessels, the gastrointestinal tract, and biliary tract, and the like by being inserted into narrowed or blocked regions without performing a surgical operation when the flow of blood or body fluids is not smooth due to occurrence of malignant or benign diseases. For example, when a blood clot occurs in a blood vessel and completely blocks a blood flow flowing to heart muscle, a heart attack may occur. In this case, a stent helps to open a coronary artery.

Meanwhile, electronic devices which are implanted and inserted into the body to acquire biological information are continuously being developed. The electronic devices are inserted into the body, collect biological information at the inserted positions, and convert the biological information into electronic information.

### SUMMARY OF THE INVENTION

Conventional electronic devices which are implanted in the body and inserted into the body to acquire biological information should perform transmission and reception, such as transmitting the acquired electronic information to the outside of the body or receiving information from the outside. However, since the conventional electronic devices are inserted into and positioned in the human body, it is difficult for the conventional electronic devices to perform information communication. Furthermore, a device for maintaining and assisting body functions and a device for performing communication of the biological information should be separately inserted into the body.

The present embodiment is directed to a technology that can maintain and assist body functions and perform biological information communication.

According to an embodiment, there is provided a stent antenna that is inserted into the body and used, including a main branch having a mesh shape, a plurality of branched branches which are branched off from the main branch, each having a mesh shape, and a feed line connected to the main branch to supply power to the stent antenna.

According to another embodiment, there is provided a stent antenna that is inserted into the body and used, including a graft, a wavy conductive element formed in the same shape as an outer circumference of the graft, and a feed line connected to one point and the other point of the stent antenna to supply power to the stent antenna.

According to still another embodiment, there is provided a biological information communication apparatus including a stent antenna including an antenna configured to receive a power signal transmitted in a wireless manner and transmit and receive data to and from an external device, a rectifier configured to rectify the power signal received by the stent antenna, and a sensor configured to operate using the power provided by the rectifier and is inserted into the body to detect the biological information of the body, wherein the stent antenna, the rectifier, and the sensor are inserted into the body and operate.

### BRIEF DESCRIPTION OF THE DRAWINGS

The above and other objects, features and advantages of the present invention will become more apparent to those skilled in the art by describing exemplary embodiments thereof in detail with reference to the accompanying drawings, in which:

FIG. 1 is a schematic diagram illustrating a stent antenna according to a first embodiment that is placed in the human body and communicates with an external device;

FIG. 2 is a schematic diagram illustrating states of a main branch and a branched branch that are included in the stent antenna according to the first embodiment and will be coupled;

FIG. 3A is a schematic diagram illustrating a helical wire element included in the main branch, and FIG. 3B is a schematic diagram illustrating a helical wire element included in the branched branch;

FIG. 4 is a schematic diagram illustrating a process of forming a main branch or a branched branch using the helical wire elements included in the main branch or the branched branch;

FIG. 5 is a schematic diagram illustrating the stent antenna according to the first embodiment;

FIG. 6 is a diagram illustrating a medical information providing system including a stent according to a second embodiment;

FIG. 7 is a schematic diagram illustrating a stent antenna according to the second embodiment;

FIGS. 8A, 8B, and 8C are schematic diagrams illustrating a process of forming the stent antenna (12);

FIG. 9 is a diagram illustrating a feed structure of the stent antenna;

FIG. 10A is a diagram illustrating simulation and analysis environments in a homogeneous environment for stent antennas according to the first and second embodiments, FIG. 10B is a diagram illustrating simulation and analysis environments in a homogeneous environment for the stent antenna according to the second embodiment, and FIG. 10C is a diagram illustrating a process of performing additional simulation in a real human model VariPose man to verify the performance of the stent antenna according to the present embodiment in a non-homogeneous environment;

FIG. 11 is a diagram illustrating a prototype of the stent antenna according to the first embodiment with a three-dimensional (3D) printed graft and a symmetrical segment of an abdominal aortic aneurysm model;

FIG. 12A is a diagram illustrating a prototype of the stent antenna according to the second embodiment that is formed on a 3D printed graft, and FIG. 12B is a diagram illustrating the prototype of the stent antenna according to the second embodiment that is inserted into an abdominal aortic aneurysm model;

FIG. 13A is a diagram illustrating an actual verification process of a stent system according to the proposed present embodiment that is measured using a model of the American Society for Testing and Materials (ASTM), which is filled with a saline solution, FIG. 13B is a diagram illustrating an environment in which measurement is performed in a container filled with minced pork, and FIG. 13C is a diagram illustrating that measurement is performed inside an anechoic chamber in which a reference antenna is installed 12 m away from a device under test (DUT);

FIG. 14 is a diagram illustrating the results of a simulation and a test of reflection coefficients of the stent antenna according to the first embodiment, which are investigated in an acoustic event detection (AED) (homogeneous model), a heterogeneous model (Remcom), an ASTM phantom, and minced pork;

FIGS. 15A and 15B are diagrams illustrating the results of a simulation and a test of reflection coefficients of the stent antenna according to the second embodiment, which are investigated in the AED (homogeneous model), the heterogeneous model (Remcom), the ASTM phantom, and the minced pork;

FIG. 16 is a diagram illustrating E-plane and H-plane gain patterns of the proposed stent antenna of the first embodiment at frequencies of 868 MHz and 915 MHz;

FIG. 17 is a diagram illustrating gain patterns of the stent antenna of the second embodiment at frequencies of 434 MHz, 868 MHz, and 915 MHz;

FIGS. 18A and 18B are diagrams illustrating current distributions of an abdominal aortic aneurysm model, and graft, blood, and stent models of the AED at frequencies of 868 MHz and 915 MHz in the stent antenna according to the first embodiment, and FIG. 18C shows diagrams illustrating electric field distributions in three plane slices, i.e., a transverse plane, sagittal plane, and coronal plane, of the stent antenna according to the first embodiment;

FIGS. 19A and 19B are diagrams illustrating current distributions of the abdominal aortic aneurysm model, and the graft, blood, and stent models of the AED at frequencies of 868 MHz and 915 MHz in the stent antenna according to the second embodiment, and FIG. 19C shows diagrams illustrating electric field distributions in the three plane slices, i.e., the transverse plane, sagittal plane, and coronal plane, of the stent antenna according to the second embodiment; and

FIGS. 20A and 20B are diagrams illustrating a current and an electric field generated by the stent antenna (10) in a real human body model.

### DETAILED DESCRIPTION OF EXEMPLARY EMBODIMENTS

### First Embodiment

Hereinafter, a first embodiment will be described with reference to the accompanying drawings.

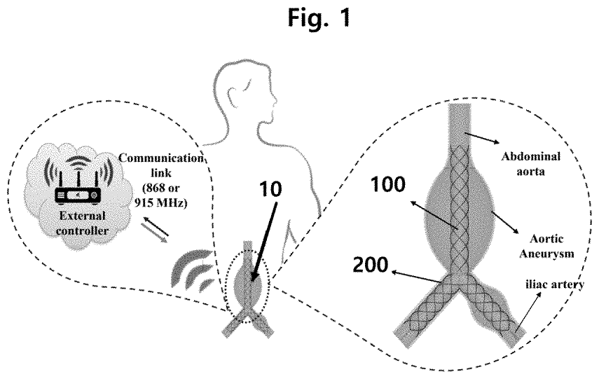

FIG. 1 is a schematic diagram illustrating a stent antenna 10 according to the first embodiment that is placed in the human body and communicates with an external device. In the illustrated embodiment, the stent antenna 10 is illustrated in a state of being inserted into the abdominal aorta and iliac arteries branching from the abdominal aorta, but this is merely an example, and a portion of the body into which the stent antenna according to the first embodiment is insertable is not limited.

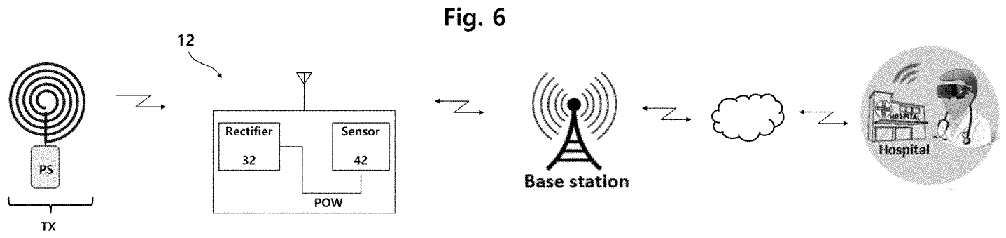

As illustrated in FIG. 1, the stent antenna 10 according to the first embodiment is inserted at a position where an aneurysm is formed and may perform a stent function and also perform an antenna function for communicating with an external telemetry device. The stent antenna 10 is inserted at the position where the aneurysm is formed and is electrically connected to a sensor (see FIG. 6) configured to detect information such as a heart rate, blood flow, and blood pressure and may receive biological information detected by the sensor. In one example, the stent antenna 10 transmits the acquired biological information to the telemetry device. In another example, the stent antenna 10 may transmit the acquired biological information to a place providing remote medical services, such as a hospital, through a base station (see FIG. 6).

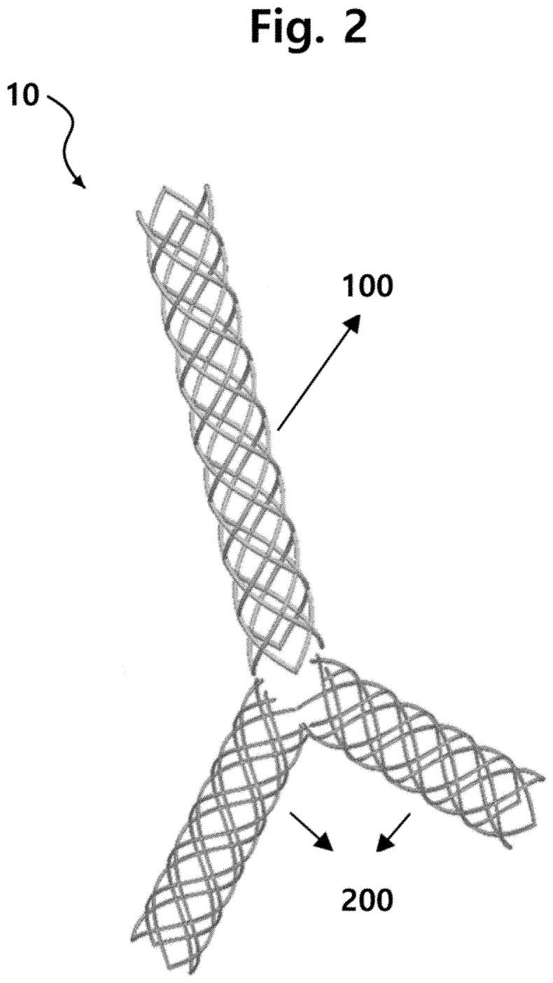

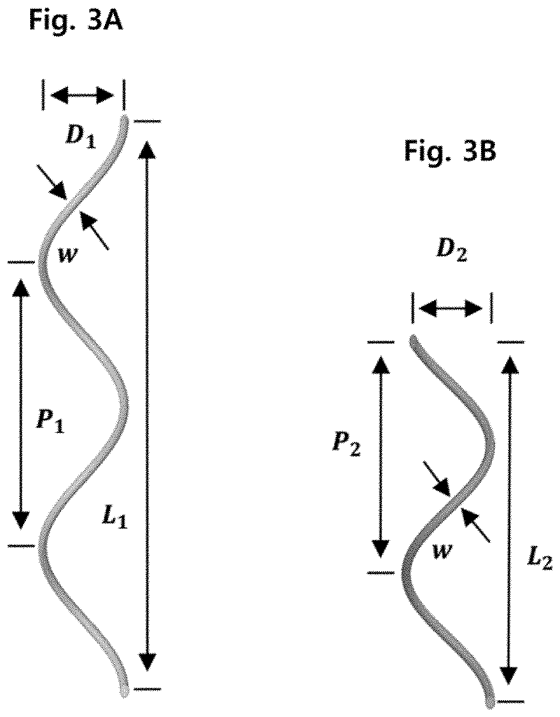

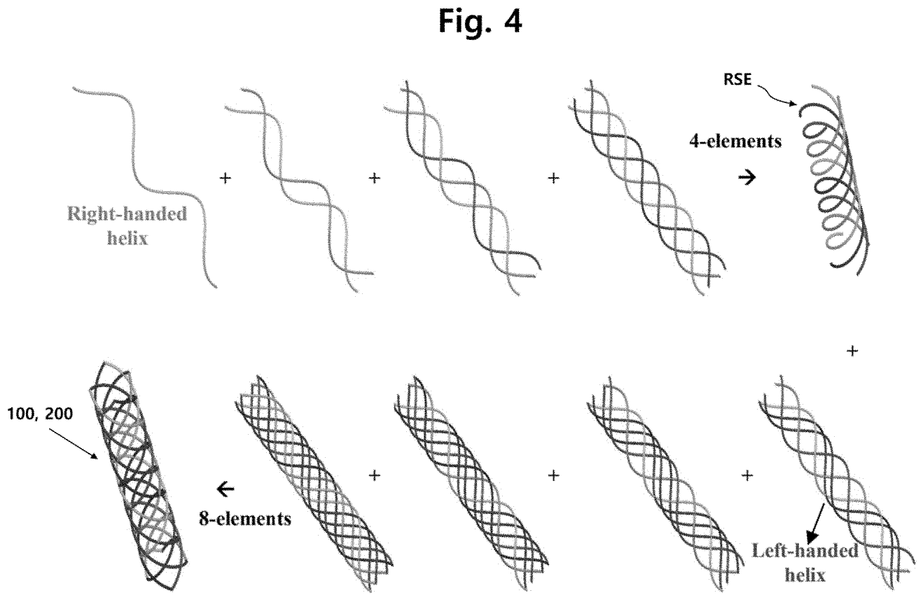

FIG. 2 is a schematic diagram illustrating states of a main branch 100 and branched branches 200 that are included in the stent antenna 10 according to the first embodiment and will be coupled. FIG. 3A is a schematic diagram illustrating a helical wire element included in the main branch 100, and FIG. 3B is a schematic diagram illustrating a helical wire element included in the branched branch 200. FIG. 4 is a schematic diagram illustrating a process of forming the main branch 100 or the branched branch 200 using the helical wire elements included in the main branch 100 or the branched branch 200.

Referring to FIGS. 2, 3A, 3B, and 4, the stent antenna 10 according to the first embodiment may include one main branch 100 and two branched branches 200. As one example, when the stent antenna 10 according to the first embodiment is implanted in the abdominal aorta, the main branch 100 may be positioned in the abdominal aorta, and the branched branches 200 may be positioned in two iliac arteries. The main branch 100 and the branched branches 200 include right-handed helical wire elements and left-handed helical wire elements, and a stent may be made of stainless steel 316L wire which is a medical material.

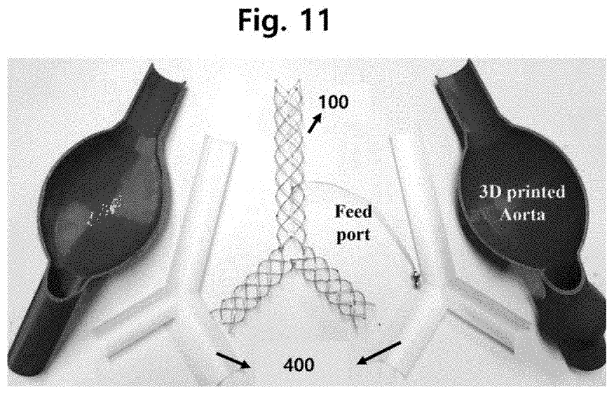

Since the stent has key material characteristics such as biocompatibility, biodegradability, and non-toxicity, the stent is disposed in a graft 400 (see FIG. 11) made of polylactic acid (PLA) material that is widely used in the development of vascular grafts, sutures, and other surgical implants. The stent may be placed inside a graft, outside a graft, or between grafts and may not be in contact with body tissue.

The main branch 100 includes four right-handed helical wire elements and four left-handed helical wire elements. Each wire element included in the main branch 100 may be a stainless steel wire of a 316L material, which has a diameter of 0.5 mm. The wire element included in the main branch 100 may have two turns in a length direction of the wire, a pitch of 60 mm, a length of 120 mm, and a helical diameter of 20 mm.

The four right-handed helical wire elements are successively placed and rotated 90° about the same axis to form a right-handed helical stent element (RSE). Then, by coupling the RSE formed of the four right-handed helical wire elements to a helical element formed of the four left-handed helical wire elements, it is possible to form the main branch 100 of the stent in which mesh rings uniformly distributed along a structure of the stent are formed.

The two branched branches 200 may each be formed by coupling four wire elements with 1.405 spiral turns to the right in the length direction to four wire elements with 1.405 spiral turns to the left in the length direction.

Like the main branch 100, in the branched branch 200, the four right-handed helical wire elements are successively placed and rotated 90° about the same axis to form four right-handed helical stent elements. Then, the four left-handed helical wire elements are successively placed and rotated 90° about the same axis to form four left-handed helical stent elements.

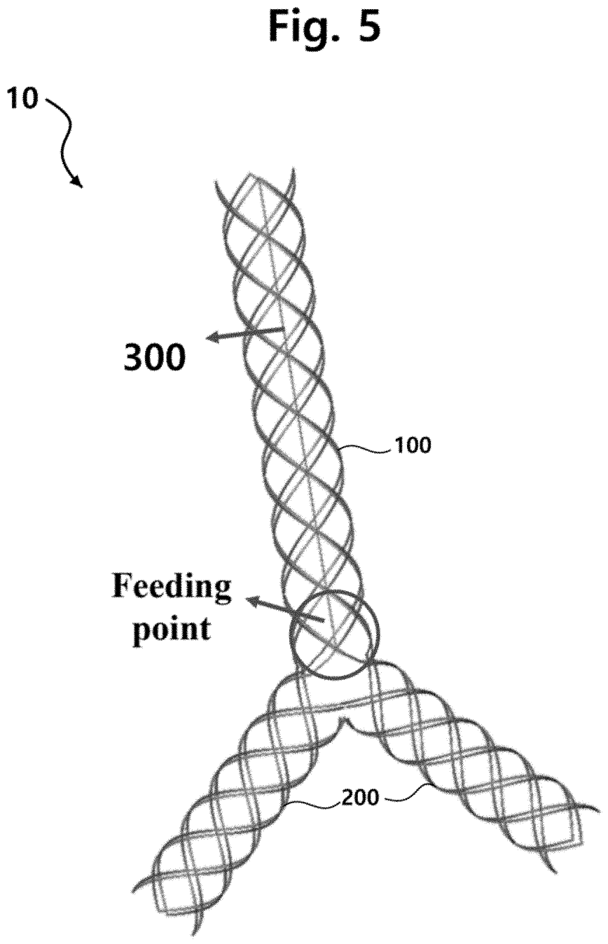

FIG. 5 is a schematic diagram illustrating the stent antenna 10 according to the first embodiment. Referring to FIG. 5, it is possible to form the stent antenna 10 having mesh rings uniformly distributed along the structure of the stent by coupling the four right-handed helical stent elements to the four left-handed helical stent elements. The stent antenna 10 according to the first embodiment may be obtained by coupling the formed main branch 100 and the two formed branched branches 200.

In the illustrated example, lengths and diameters of the main branch 100 and the branched branch 200 are varied according to the site where the stent is placed. For example, as illustrated in FIGS. 3A and 3B, when used for an abdominal aortic aneurysm, parameters of each wire element are shown in the following table.

TABLE 1

| | value | | value | | value |
|---|---|---|---|---|---|
| parameter | (mm) | parameter | (mm) | parameter | (mm) |
| P₁ | 60 | L₁ | 120 | D₁ | 20 |
| P₂ | 50 | L₂ | 70.25 | D₂ | 18.8 |
| w | 0.5 | — | — | — | — |

In the stent antenna 10 according to the first embodiment, the lengths and the diameters of the main branch 100 and the branched branch 200 correspond to a size and a length of the aneurysm. Thus, instead of designing an antenna using a standard equation, an operating frequency is analyzed by exciting the stent at a feed point which may resonate in a target frequency band.

In the example illustrated in FIG. 5, the stent antenna 10 is supplied with power by a feed line 300. As one example, the feed line 300 may be a copper wire. An end portion of the stent antenna 10 is coupled to the feed line 300. In the first embodiment, one end portion of the feed line 300 is connected to an upper portion of the stent antenna 10 and the other end thereof is connected to a lower middle portion of the stent antenna 10 where the two branched branches 200 branch from each other at an angle of 90°, thereby forming a feed point. An operating frequency may be controlled by supplying a power supply signal to the feed line 300 and changing a position of the feed point.

The operating frequency of the stent antenna 10 may be determined from a dip point observed in a reflection coefficient of the stent by analyzing different feed points according to impedance matching and the position of the feed line 300 attached to the stent antenna 10. In order to investigate the resonance of the stent antenna 10, an impedance phase dip method is used to investigate an operating frequency of a coronary artery stent from a dip point of an impedance phase of the stent.

A current path is extended such that a current flows in the length direction of the stent antenna 10 using the feed line 300. Since several loops capable of shortening the current path may be formed in the stent antenna 10, the stent antenna 10 may not operate within a target frequency band. Therefore, the power supply signal is provided through the feed line 300 instead of being provided to the stent itself so as to operate within the target frequency band.

The stent antenna 10 according to the first embodiment is supplied with power using an impedance matching point as a feed point (see FIG. 5). Thus, the stent antenna 10 may exhibit a broadband characteristic operating at a frequency in the range of 850 MHz to 950 MHz including the industrial, scientific, medical (ISM) band (i.e., 868 MHz and 915 MHz). The stent antenna 10 is placed inside the graft 400 of the stent (biocompatible polymer) (see FIG. 11), which serves as an artificial aortic wall inside an aortic aneurysm, and thus the graft 400 covers a metal portion so that the metal portion is not brought into contact with the surrounding tissue.

### Second Embodiment

Hereinafter, a second embodiment will be described with reference to FIGS. 6 to 9. FIG. 6 is a diagram illustrating a medical information providing system including a stent antenna 12 according to a second embodiment. Referring to FIG. 6, the stent antenna 12 is placed in the body and serves as a stent and an antenna. The stent antenna 12 operates at frequencies in at least two different bands, receives power from a wireless power transmitter TX in a wireless manner within one band, and transmits and receives biological information to and from a base station at frequencies in another band. The biological information received by the base station may be provided to a place providing remote medical services (for example, a hospital). As one example, a frequency band for receiving power in a wireless manner may be a frequency band ranging from 863 MHz to 870 MHz (868 MHz), and a frequency band for transmitting and receiving data may be a frequency band ranging from 902 MHz to 928 MHz (915 MHz).

In one example, the stent antenna 12 may further include a rectifier 32 configured to receive and rectify a power signal of a radio frequency (RF) band, and a sensor 42 placed inside the body to collect biological information such as a blood pressure, a heart rate, and a body temperature. The rectifier 32 generates power for driving the sensor 42 from the received power signal and provides the power to the sensor 42.

The sensor 42 receives power from the rectifier 32 and collects the biological information from the body. The collected biological information is provided to the base station through a communication unit (not shown) and the stent antenna 12 connected to the communication unit. For example, a frequency band through which the stent antenna 12 transmits and receives information to and from the base station may be a 915 MHz band. Both the 868 MHz band, which is the above-described wireless power transmission frequency band, and the 915 MHz band, which is the above-described frequency band for transmitting and receiving information, may be frequencies of the ISM band. However, alternatively, any one or more of the above-described frequencies may be changed according to embodiments.

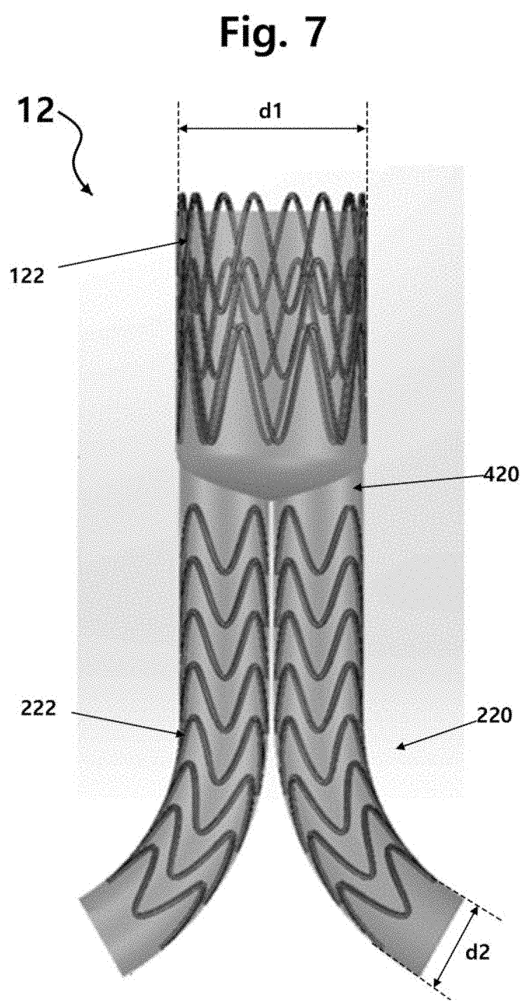

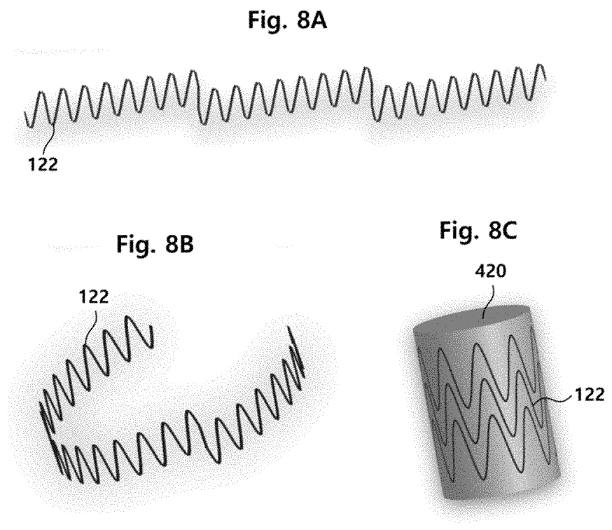

FIG. 7 is a schematic diagram illustrating the stent antenna 12 according to the second embodiment. FIG. 8 is a diagram for describing a structure of the stent antenna 12. Referring to FIGS. 7 and 8, the stent antenna 12 according to the second embodiment includes a graft 420, a wavy conductive element 122 formed in the same shape as an outer circumference of the graft 420, and a feed line connected to one point and the other point of the stent antenna 12 to supply power to the stent antenna 12.

The stent antenna 12 according to the second embodiment may include two branched branch stents 220. As shown in FIG. 7, the stent antenna 12 and the branched branch stents 220 are not electrically connected to each other, and the two branched branch stents 220 are also not electrically connected to each other. However, due to a coupling effect, a current may also flow in the two branched branch stents 220.

As in the above-described embodiment, when the stent antenna 12 according to the second embodiment is implanted in the abdominal aorta, the stent antenna 12 may be placed in the abdominal aorta (see FIG. 1), and the branched branch stents 220 may be placed in two iliac arteries (see FIG. 1).

FIGS. 8A, 8B, and 8C are schematic diagrams illustrating a process of forming the stent antenna 12, and FIG. 8A is a schematic diagram illustrating the conductive element 122 constituting the stent antenna 12. As one example, the conductive element 122 may be a conductive metal, for example, a stainless wire. As shown in the drawings, the conductive element 122 may be processed into the form of a wavy shape.

Referring to FIG. 8B, the wavy conductive element 122 is processed to correspond to a shape of the outer circumference of the graft 420, and as shown in FIG. 8C, the stent antenna 12 is placed on the graft 420. According to an embodiment not shown in the drawings, the wavy conductive element 122 is placed on the outer circumference of the graft and is covered with a graft cover so as to not be exposed so that the conductive element 122 is prevented from being in contact with the body and/or a body fluid such as blood.

Referring to FIGS. 7 and 8, as shown in the drawings, the stent antenna 12 may include the wavy conductive element 122, and the branched branch stents 220 may also include second wavy conductive elements 222. As one example, the stent antenna 12 may be formed of a single conductive element 122, and each of the branched branch stents 220 may be formed of a single second conductive element 222. In the illustrated embodiment, a diameter d1 of the stent antenna 12 may be greater than a diameter d2 of the branched branch stent 220.

The conductive element 122 and the branched branch stent 220 constituting the stent antenna 12 and the second conductive element 222 are placed on the graft 420. The graft 420 may include any one material of PLA and polytetrafluoroethylene (PTFE) which have key material characteristics such as biocompatibility, biodegradability, and non-toxicity and are widely used in the development of vascular grafts, sutures, and other surgical implants. The stent antenna 12 may be placed inside a graft, outside a graft, or between grafts and may not be in contact with body tissue.

When the stent antenna 12 according to the present embodiment is inserted into the abdominal aorta, the diameter d1 of the stent antenna 12 may range from 28 mm to 30 mm, and the diameter d2 of the branched branch stent 22022 may range from 12 mm to 14 mm. In addition, a length of the main branch 122 may range from 35 mm to 40 mm, and a length of the branched branch 220 may range from 58 mm to 62 mm. As one example, each wire element included in the main branch 120 and the branched branch stent 220 may be a stainless steel wire of a 316L material, which has a diameter of 0.5 mm.

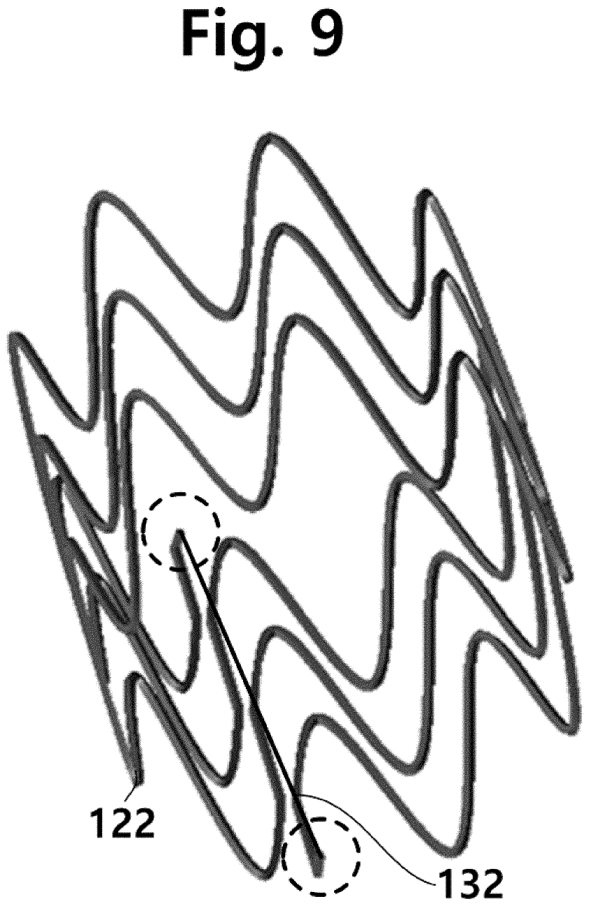

FIG. 9 is a diagram illustrating a feed structure of the stent antenna 12. Referring to FIG. 9, the stent antenna 12 may be supplied with power by a feed line 132. In the illustrated example, the feed line 132 may be a copper line. One end and the other end of the feed line 132 may be connected to different positions of the conductive element 122 constituting the stent antenna 12. As in the illustrated example, one end of the feed line 132 may be connected to one end portion of the conductive element 122 constituting the stent antenna 12, and the other end thereof may be connected to the other end portion of the conductive element 122. An operating frequency may be controlled by supplying a power supply signal to the feed line 132 and changing a position of a feed point. This is the same as in the above-described first embodiment. In another example, the feed line may be a coaxial cable.

The operating frequency of the stent antenna 12 may be determined from a dip point observed in a reflection coefficient of the stent by analyzing different feed points according to impedance matching and a position of the feed line 132 attached to the stent antenna 12. In order to investigate the resonance of the stent antenna 12, an impedance phase dip method is used to investigate an operating frequency of a coronary artery stent from a dip point of an impedance phase of the stent.

Since several loops for shortening a current path may be formed in the stent antenna 12, the stent antenna 12 may not operate within a target frequency band. However, according to the present embodiment, since a current flows along the conductive element 122 constituting the stent antenna 12, radio waves are emitted at a target frequency. Therefore, it is possible to control a frequency at which the stent antenna 12 operates by controlling feed points.

The stent antenna 12 according to the present embodiment is supplied with power using an impedance matching point as a feed point (see FIG. 9). Thus, the stent antenna 12 may exhibit a broadband characteristic operating at a frequency in the range of 850 MHz to 950 MHz including the ISM band (i.e., 868 MHz and 915 MHz). As one example, the 868 MHz band may be a band used for wireless power transmission, and the 915 MHz band may be a band used for data transmission. In addition, the stent antenna 12 according to the second embodiment may operate at frequencies in a 2.45 GHz band and a fifth-generation (5G) band ranging from 3.5 GHz to 6 GHz.

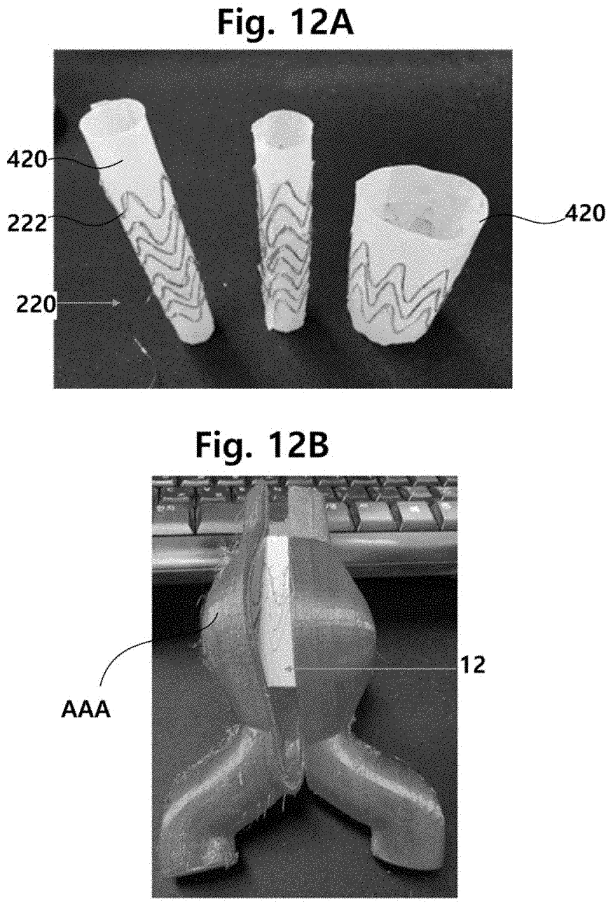

The stent antenna 12 is placed inside the graft 420 of the stent (biocompatible polymer) (see FIG. 12A), which serves as an artificial aortic wall inside an aortic aneurysm, and thus the graft 420 covers a metal portion so that the metal portion is not brought into contact with the surrounding tissue.

Setting Test and Simulation Environments

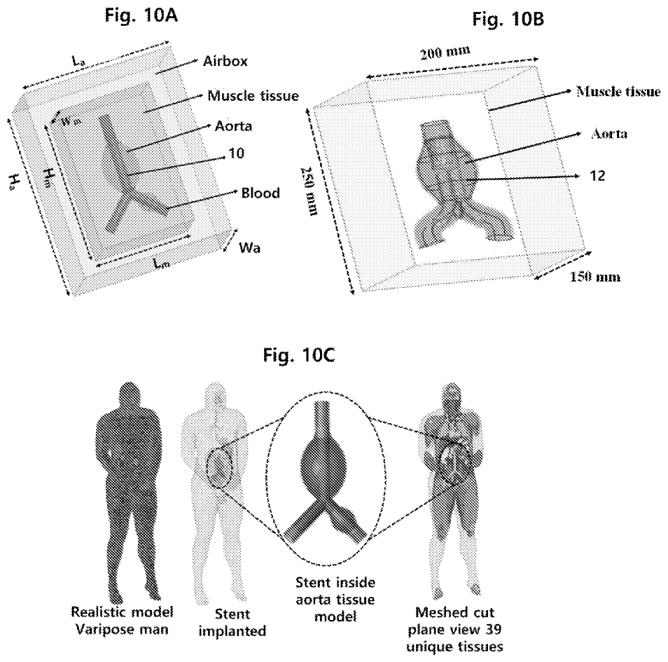

FIGS. 10A and 10B are diagrams illustrating simulation and analysis environments in a homogeneous environment for the stent antennas according to the first and second embodiments, and FIG. 10C is a diagram illustrating a process of performing additional simulation in a real human model VariPose man to verify the performance of the stent antenna according to the present embodiment in a non-homogeneous environment.

Referring to FIGS. 10A, 10B, and 10C, the proposed stent antennas 10 and 12 were designed, simulated, and analyzed using a finite element method (FEM)-based electromagnetic (EM) calculation tool Ansys Electronics Desktop (AED) and a finite difference time domain (FDTD) method (XFDTD)-based Remcom. As shown in FIG. 10A, for simplicity of calculation, an initial simulation of the stent antenna 10 of the first embodiment was performed on a homogeneous muscle phantom (HMP) modeled as a single layer box of 200×200×300 mm in the AED. In addition, as shown in FIG. 10B, an initial simulation of the stent antenna 12 of the second embodiment was performed on an HMP modeled as a single layer box of 250×200×150 mm.

In order to implement blood vessels in the HMP, in a tissue model of the abdominal aortic aneurysm model, the blood vessel was designed to include blood tissue and have a thickness of 2 mm. The blood vessel was modeled as a cylinder connected to the aorta and both iliac arteries. Frequency dependent properties of tissues in both ISM bands were collected from an open source standard database for human body dielectric properties and are summarized in the following table.

TABLE 2
HUMAN BODY DIELECTRIC PROPERTIES

| | 868 MHz | | 915 MHz | |
|---|---|---|---|---|
| | Permittivity | Conductivity | Permittivity | Conductivity |
| Blood | 61.5 | 1.52 S/m | 61.3 | 1.54 S/m |
| Aorta | 44.9 | 0.686 S/m | 44.7 | 0.701 S/m |
| MusCle | 55.1 | 0.932 S/m | 55 | 0.948 S/m |

The stent antenna according to the present embodiment was placed inside the blood tissue model assuming a blood flow of the blood vessel model of the abdominal aortic aneurysm model surrounded by the HMP, and the stent antenna was placed at a depth of 100 mm in a center of the HMP.

As shown in FIG. 10C, in order to verify the performance of the stent antennas 10 and 12 according to the present embodiment in a non-homogeneous environment, an additional simulation was performed on a real human model VariPose man composed of 39 unique tissues that are usable in Remcom. Since a tissue voxel model is recognized as including a normal blood vessel without an aneurysm in the abdominal aorta, the abdominal aortic aneurysm model and a tissue model of blood were introduced in the homogeneous environment as illustrated in FIG. 10A.

The FEM-based AED and the XFDTD-based Remcom were used to calculate a reflection coefficient, a far-field radiation pattern, an electric field distribution, a specific absorption rate (SAR) safety level, and maximum input power.

FIG. 11 is a diagram illustrating a prototype of the stent antenna 10 according to the first embodiment with a three-dimensional (3D) printed graft 400 and a symmetrical segment of an abdominal aortic aneurysm model AAA. FIG. 12A is a diagram illustrating a prototype of the stent antenna 12 according to the second embodiment that is formed on a 3D printed graft 420, and FIG. 12B is a diagram illustrating the prototype of the stent antenna 12 according to the second embodiment that is inserted into the abdominal aortic aneurysm model AAA. As shown in FIGS. 11, 12A, and 12B, the prototypes were manufactured and the performance of the stent antennas 10 and 12 according to the present embodiment was measured.

In the stent antenna 10 of the first embodiment, all the wire elements were soldered at a junction of the main branch and the branched branches and at a crown point of the end portions thereof to form a stent. The stent antenna 10 was formed by connecting the main branch 100 and the branched branches 200.

The abdominal aortic aneurysm model AAA and the 3D model of the graft 400 were also printed due to a large print volume provided by FlashForge Guider II capable of printing the abdominal aortic aneurysm model and half of the graft 400 as a single printed material.

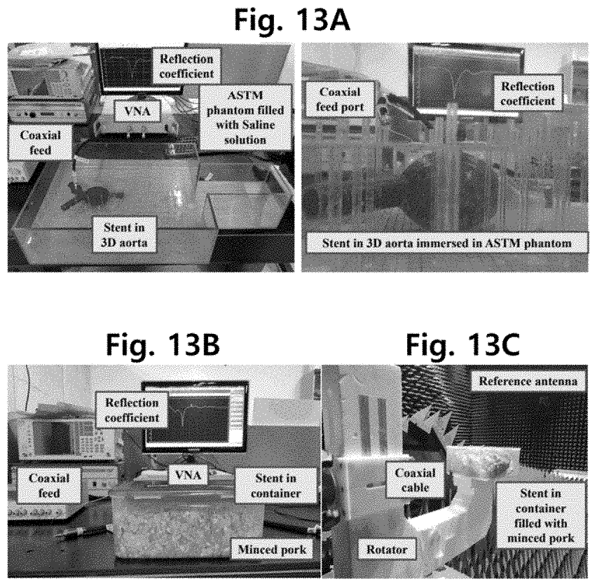

FIG. 13A is a diagram illustrating an actual verification process of a stent system according to the proposed embodiment, which is measured and guaranteed using an ASTM phantom filled with a saline solution. The proposed stent antenna and the abdominal aortic aneurysm model were completely immersed in the ASTM phantom filled with a saline solution.

As shown in FIG. 13B, measurement of test results was performed in a container filled with minced pork. In the case of the minced pork, in order to mimic blood tissue, the abdominal aortic aneurysm model was filled with a saline solution and covered with commercially available polyethylene plastic to block fluid leakage.

A vector network analyzer was used to measure a reflection coefficient of the stent in the ASTM model and the minced pork. In order to supply power to the prototype of the stent antenna, a coaxial feed probe was connected to the feed line.

As shown in FIG. 13C, a far-field radiation pattern was measured inside an anechoic chamber in which a reference antenna was installed 12 m away from a DUT. In the minced pork, the stent antenna according to the present embodiment was placed on a platform as a DUT and rotated at a specified angle by a rotator.

Rotation of the tissue container may leak a saline solution into the surrounding test setup in the abdominal aortic aneurysm model and damage the measurement system. Therefore, in order to prevent saline from leaking, polyethylene plastic was tightly wrapped around the abdominal aortic aneurysm model and then a test procedure proceeded.

Link Budget Analysis of Stent

In order to adjust device parameters, transmit stored information, and perform real-time transmission of a vital sign, a biotelemetry link is used, an implantable device serves as a transmitter, and an external interrogator serves as a receiver. Regardless of transmission power limitations, an antenna of an implantable device should provide a radio signal of sufficient strength to allow an external controller to readily receive all signals.

In order to evaluate far-field biotelemetry communication with the external interrogator, link budget analysis was performed on the stent antenna 10 according to the first embodiment and the stent antenna 12 according to the second embodiment. The availability of wireless data communication is determined by a link margin which is determined by calculating a difference in power between an available link and a necessary link. In addition, the link margin also includes several power loss factors such as antenna mismatch loss, path loss, cable and connector loss, and material loss. Generally, a zero dB link margin is considered to be effective. This means that an available link exceeds a margin of a necessary link and can be used for wireless transmission of biotelemetry data.

However, in the evaluation of the present embodiment, in order to secure better and more stable biotelemetry communication between the proposed stent antenna 10 according to the first embodiment, the proposed stent antenna 12 according to the second embodiment, and a communicator, a 10 dB link margin was considered as a minimum margin. By using parameters of standard equations (1), (2), (3), (4), and (5) exemplified by the following equations, a link budget was calculated, and the results are shown in Table 3.

$$[ Equation 1 ]$$

$$Available Link C / {N}_{0} = {P}_{t} - 2 {L}_{feed} + {G}_{t} - {L}_{f} \qquad ( 1 )$$

$$Required Link C / {N}_{0} = {E}_{b} / {N}_{0} + 10 {log}_{10} {B}_{r} - {G}_{c} + {G}_{d} \qquad ( 2 )$$

$${L}_{f} ( dB ) = 20 {log}_{10} ( \frac{4 π d}{λ} ) \qquad ( 3 )$$

$${N}_{0} = 10 {log}_{10} k + 10 {log}_{10} {T}_{i} \qquad ( 4 )$$

$${T}_{i} = {T}_{0} ( NF - 1 ) \qquad ( 5 )$$

Results

It was predicted that the stent according to the present embodiment could serve as an antenna. The performance of each of the stent antennas according to the first and second embodiments is analyzed in relation to antenna characteristics. For this reason, a reflection coefficient, a radiation pattern, a current and electric field distribution, patient safety, and wireless biotelemetry of the stent antenna system according to the present embodiment were analyzed.

A. Measurement of Reflection Coefficient |S11|

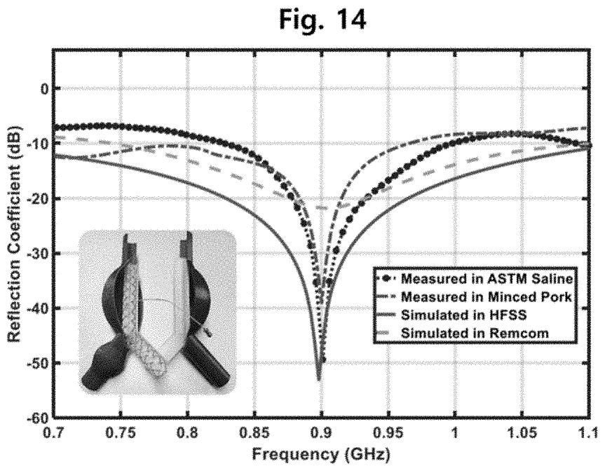

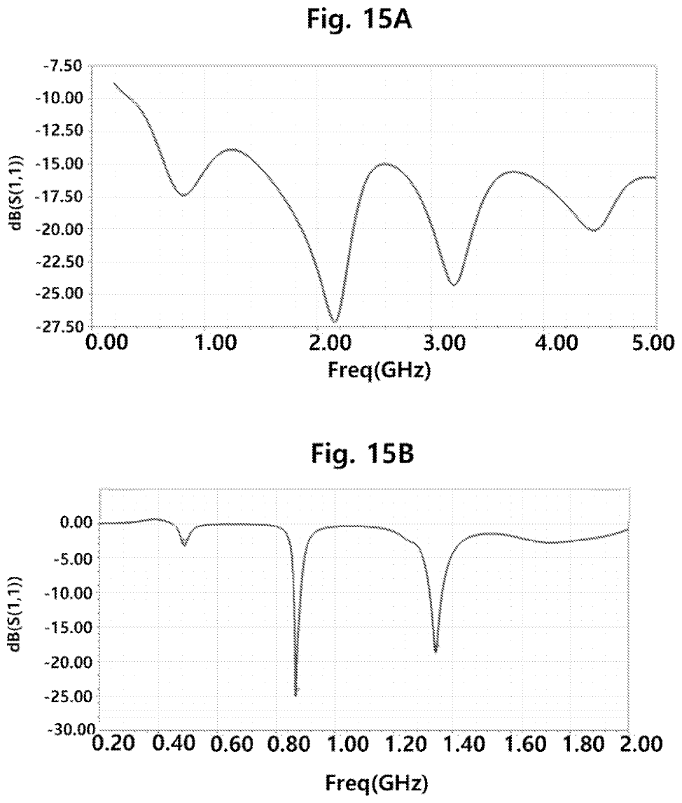

FIGS. 14, 15A, and 15B are diagrams illustrating the results of a simulation and a test of reflection coefficients of the stent antenna 10 according to the first embodiment and the stent antenna 12 according to the second embodiment, which are investigated in the AED (homogeneous model), the heterogeneous model (Remcom), the ASTM phantom, and the minced pork. The left inset in FIG. 14 shows a cutaway view of a final prototype of the proposed endovascular aneurysm repair (EVAR) stent placed in the 3D printed abdominal aortic aneurysm model and the graft model, and the final prototype was used to perform measurement. FIGS. 15A and 15B are the simulation and measured results of the reflection coefficients of the stent antenna 12 according to the second embodiment, which are investigated in the same environment.

To describe the measured results, the graphs shown in FIGS. 14, 15A, and 15B show the broadband performance of each of the first and second stent antennas and show that the stent antenna 12 serves as an antenna and operates at frequencies in two ISM bands targeted in the present embodiment, that is, in a band including frequencies of 868 MHz and 915 MHz.

The stent antenna according to the first embodiment provides a −10 dB bandwidth at a frequency of 400 MHz (700 to 1100 MHz) in the homogeneous environment, at a frequency of 360 MHz (740 to 1100 MHz) in the heterogeneous environment, at a frequency of 185 MHz (785 to 970 MHz) in the minced pork, and at a frequency of 175 MHz (825 to 1000 MHz) in the ASTM phantom.

In the measurement environment, the bandwidth of the stent antenna 10 according to the first embodiment was lower than that in the simulation environment, and the stent antenna 10 consistently operated in the two ISM bands including the frequencies of 868 MHz and 915 MHz. It is understood that the cause of the above difference is that the stent antenna 10 according to the present embodiment was more accurately modeled and designed in the simulation environment.

In contrast, the prototype of the stent antenna 10 according to the present embodiment manufactured in the measurement environment was affected by a manufacturing tolerance and inaccuracy in the prototype design. That is, since the stent antenna 10 was manually formed by winding the wire around the 3D molding mold, the wire may not be smoothly wound around the 3D printing mold. Therefore, unlike the simulation model, there was a possibility that the mesh rings were not uniformly distributed in the length direction of the stent prototype.

Nevertheless, in all cases, the stent antenna 10 exhibited performance of less than −20 dB in terms of the reflection coefficient, indicating that effects of the bandwidth difference and the manufacturing inaccuracy were overcome. The result of the reflection coefficient measured in the present embodiment is consistent with the result of the reflection coefficient |S11| of the state-of-the-art implantable antenna.

B. Measurement of Radiation Pattern and Gain

To achieve reliable biotelemetry communication, a far-field gain of the antenna is an important factor indicating receiver sensitivity required for successful information exchange. Generally, since the range of wireless biotelemetry increases, a high-gain implantable antenna is advantageous. However, since the human body is composed of several lossy tissues, the gain of the implantable antenna is inevitably degraded.

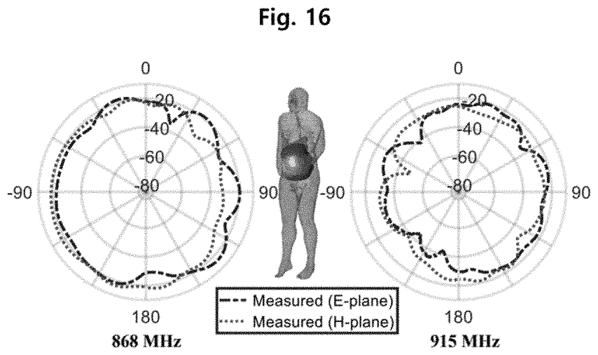

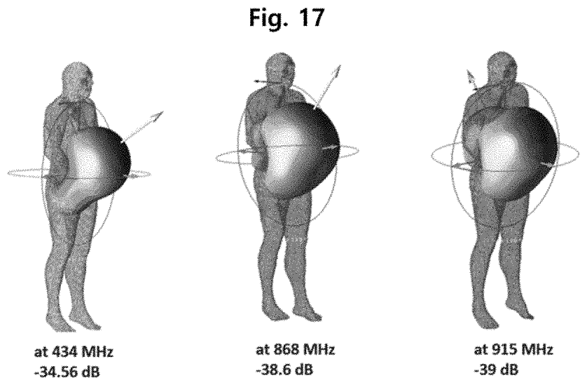

FIG. 16 is a diagram illustrating E-plane and H-plane gain patterns of the proposed stent antenna 10 of the first embodiment at frequencies of 868 MHz and 915 MHz, and FIG. 17 is a diagram illustrating gain patterns of the stent antenna 12 of the second embodiment at frequencies of 434 MHz, 868 MHz, and 915 MHz. Referring to FIG. 16, the stent antenna 10 of the first embodiment has measured peak gain values of −20.75 dBi and −19.04 dBi at frequencies of 868 MHz and 915 MHz, respectively. Referring to FIG. 17, the stent antenna 12 of the second embodiment has measured peak gain values of −34.56 dBi, −38.6 dBi, and −39 dBi at frequencies of 434 MHz, 868 MHz, and 915 MHz, respectively.

From the illustrated examples, it can be seen that the stent antennas provide omnidirectional beam patterns in both bands of inhomogeneous and irregular anatomical tissues.

C. Stent Current and Electric Field Distributions

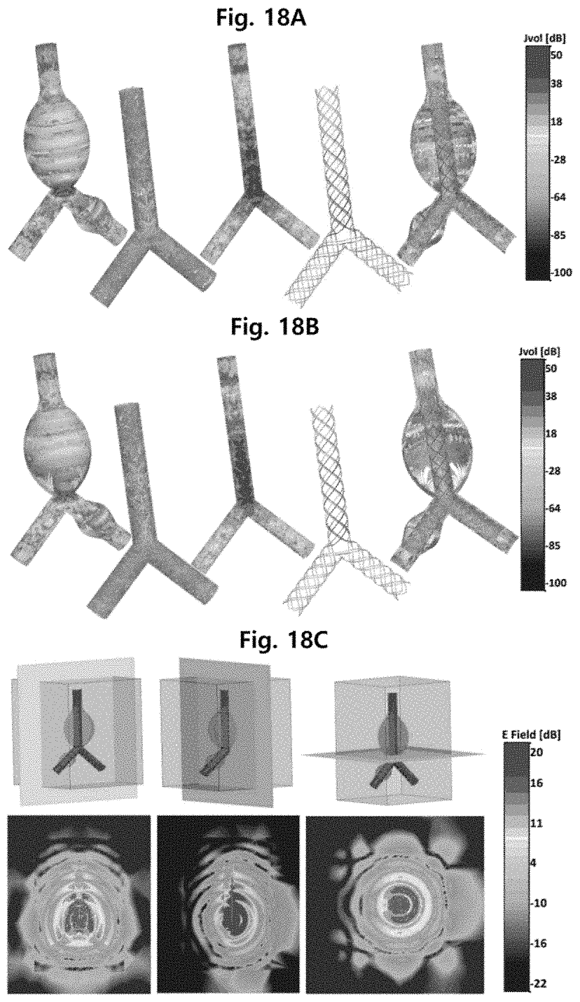

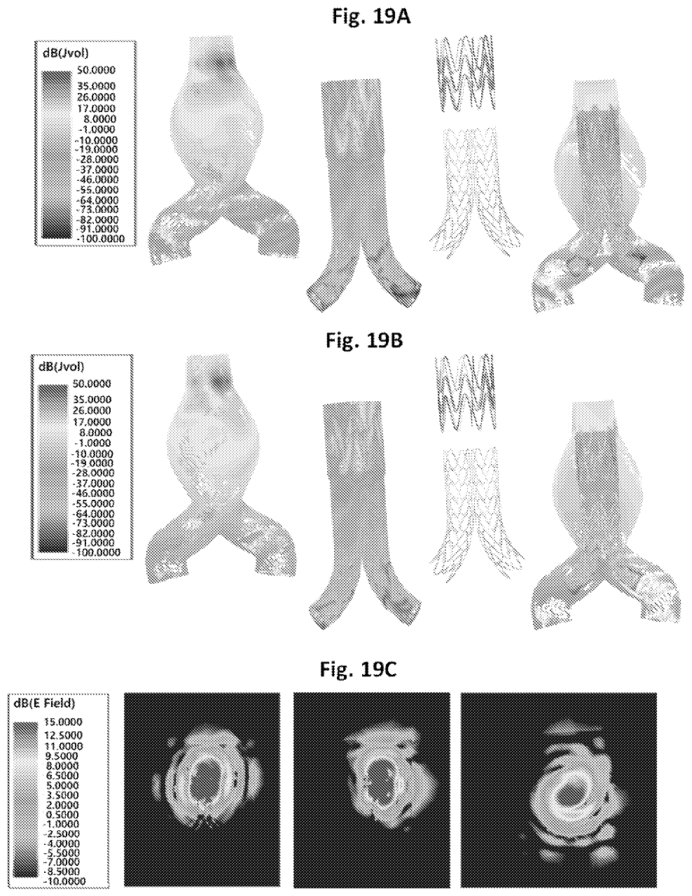

In order to analyze current and electric field distributions of the stent antennas, the stent antenna according to the first embodiment and the stent antenna according to the second embodiment were simulated in homogeneous (AED) and non-homogeneous (Remcom) environments. FIGS. 18A and 18B are diagrams illustrating current distributions of the abdominal aortic aneurysm model, and the graft, blood, and stent models of the AED from left to right in the stent antenna according to the first embodiment. FIGS. 19A and 19B are diagrams illustrating current distributions of the abdominal aortic aneurysm model, and the graft, blood, and stent models of the AED from left to right in the stent antenna according to the second embodiment. FIGS. 18C and 19C are diagrams illustrating electric field distributions in three plane slices, i.e., a transverse plane, a sagittal plane, and a coronal plane of each of the stent antenna according to the first embodiment and the stent antenna according to the second embodiment.

A current flow is mainly formed in the length direction of the stent and electromagnetic (EM) energy may be emitted at frequencies of 868 MHz and 915 MHz. The current distributions according to the components of the integrated stent antenna model are clearly shown in the cross-sectional views shown on the rightmost side of FIGS. 18A, 18B, 19A, and 19B.

As shown in FIGS. 19A and 19B, the most remarkable aspect is that, although the branched branch stent is not electrically connected to the stent antenna, a current flows due to a coupling effect. The current of the stent antenna is affected by the mutual coupling of the feed line as well as a supply end. According to the current distribution analysis at the frequency of 915 MHz, both the feed line and the metal stent serve as an antenna. Since a mesh loop is present according to a stent length restricting a current flow when the feed line is not present, excitation of the stent may be difficult. For example, when excitation is provided directly at a specific point of the stent, since a very limited current flow is present at the surface of the stent, a greater reflection is provided so that many EM waves are reflected due to imperfect impedance matching. For this reason, the stent antenna is supplied with power by the feed line.

FIGS. 18C and 19C are diagrams illustrating electric field distributions in the three plane slices, i.e., the transverse plane, the sagittal plane, and the coronal plane. A plot of a homogeneous medium shows a forward electric field pattern represented by the stent starting at a center of the stent in the vicinity of an excitation point corresponding to a dipole mode.

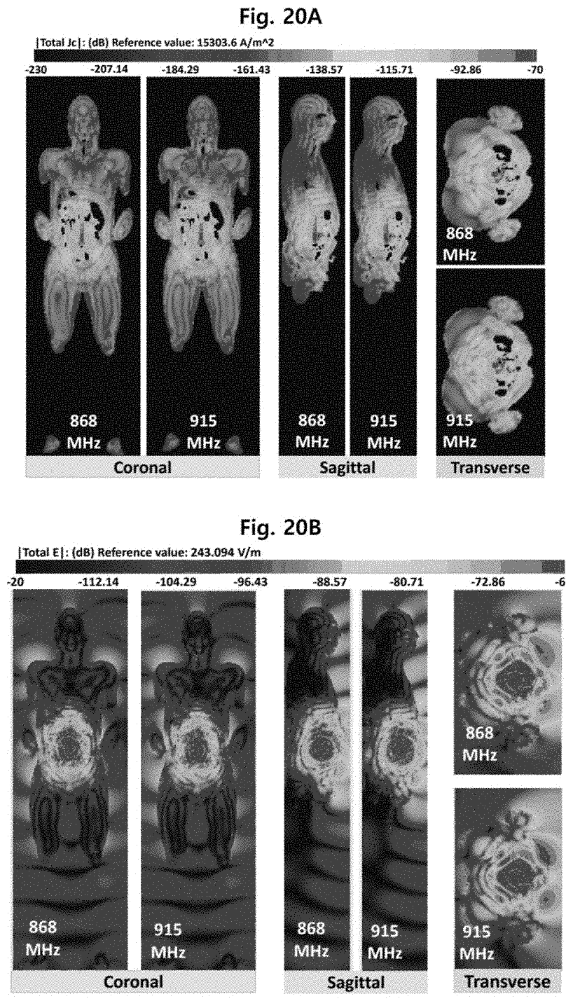

FIGS. 20A and 20B are diagrams illustrating a current and an electric field generated by the stent antenna 10 in a real human body model. The electric field is dominant around the stent antenna, and this indicates that the distributions are well matched in both homogeneous and heterogeneous cases and that the stent antennas operate well in both homogeneous and heterogeneous environments.

In accordance with the present embodiment, a stent antenna has an advantage of serving as a stent inserted into the body and serving as an antenna for performing communication with an external communication device.

In order to aid understanding of the present invention, the description has been made with reference to embodiments shown in the drawings, but these embodiments are for implementation and are merely illustrative. Thus, those skilled in the art will appreciate that various modifications and equivalent other embodiments can be derived without departing from the scope of the present invention. Therefore, the true technical scope of the present invention should be defined by the appended claims.

This work was supported by the Institute of Information and Communications Technology Planning and Evaluation (IITP) Grant funded by the Korean Government Ministry of Science and ICT (MIST), under Grant 2022-0-00310.

What is claimed is:

1. A stent antenna that is inserted into a body and used, comprising:
a main branch having a mesh shape;
a plurality of branched branches which are branched off from the main branch and each have a mesh shape; and
a feed line connected to the main branch to supply power to the stent antenna.

2. The stent antenna of claim 1, wherein the main branch and the branched branches are each made of a plurality of metal wires.

3. The stent antenna of claim 2, wherein the mesh shape includes:
a right-handed helical wire element formed by causing the metal wire to have right turns in a length direction of the metal wire; and
a left-handed helical wire element formed by causing the metal wire to have left turns in the length direction of the metal wire.

4. The stent antenna of claim 3, wherein a pitch of each of the right-handed helical wire element and the left-handed helical wire element included in the main branch is greater than a pitch of each of the right-handed helical wire element and the left-handed helical wire element included in the branched branch.

5. The stent antenna of claim 3, wherein:
the right-handed helical wire element is formed by coupling two or more metal wires which have right turns in the length direction; and
the left-handed helical wire element is formed by coupling two or more metal wires which have left turns in the length direction.

6. The stent antenna of claim 3, wherein mesh rings formed by the right-handed helical wire element and the left-handed helical wire element are uniformly distributed in the stent antenna.

7. The stent antenna of claim 2, wherein the metal wire includes a stainless steel wire.

8. The stent antenna of claim 1, wherein a diameter of the main branch is greater than a diameter of the branched branch.

9. The stent antenna of claim 1, wherein the stent antenna further includes a graft disposed so that the main branch and the branched branches are not in contact with the body.

10. The stent antenna of claim 9, wherein the stent antenna is placed inside the graft, and the stent antenna and the graft are both inserted into the body.

11. The stent antenna of claim 9, wherein the graft is formed of polylactic acid (PLA).

12. The stent antenna of claim 1, wherein:
one end of the feed line is connected to any one of the main branch and the branched branch to form a feed point; and
the other end of the feed line is connected to an end portion of the main branch.

13. The stent antenna of claim 1, wherein the stent antenna operates at frequencies in at least two different bands within an industry-science-medical (ISM) band.

14. The stent antenna of claim 1, wherein:
the stent antenna is installed in the abdominal artery and iliac arteries of the body;
the main branch is installed in the abdominal artery; and
the branched branches are installed in the iliac arteries.

15. A stent antenna that is inserted into a body and used, comprising:
a graft;
a wavy conductive element formed in the same shape as an outer circumference of the graft; and
a feed line connected to one point and the other point of the stent antenna to supply power to the stent antenna.

16. The stent antenna of claim 15, wherein the stent antenna is made of a single conductive element.

17. The stent antenna of claim 15, wherein the conductive element includes a metal wire.

18. The stent antenna of claim 15, wherein:
the stent antenna and the graft are both inserted into the body; and
the stent antenna is not in direct contact with the body.

19. The stent antenna of claim 15, wherein the graft is formed of any one material of polylactic acid (PLA) and polytetrafluoroethylene (PTFE).

20. The stent antenna of claim 15, wherein an operating frequency of the stent antenna is determined by a length of the conductive element between the one point and the other point.

21. The stent antenna of claim 15, wherein the stent antenna operates at frequencies in at least two different bands within an industry-science-medical (ISM) band.

22. The stent antenna of claim 21, wherein one of the at least two different bands is a band for wireless data communication, and the other is a band for wireless power transmission.

23. The stent antenna of claim 15, wherein the stent antenna is installed in the abdominal artery of the body.

24. The stent antenna of claim 23, wherein:
the stent antenna further includes a branched branch stent installed in an iliac artery; and
the branched branch stent is not electrically connected to the stent antenna.

25. The stent antenna of claim 21, wherein the stent antenna includes:
a rectifier configured to receive and rectify a wireless power signal at a frequency in any one band of the at least two different bands and provide the rectified wireless power signal; and
a sensor driven by power provided by the rectifier and configured to collect biological information,
wherein the stent antenna transmits the biological information collected by the sensor through any one band of the at least two different bands.

26. A biological information communication apparatus comprising:
a stent antenna including an antenna configured to receive a power signal transmitted in a wireless manner and transmit and receive data to and from an external device;
a rectifier configured to rectify the power signal received by the stent antenna; and
a sensor configured to operate using the power provided by the rectifier and is inserted into a body to detect biological information of the body,
wherein the stent antenna, the rectifier, and the sensor are inserted into the body and operate.
